(19) United States
(12) Patent Application Publication
(10) Pub. No.: US 2008/0223789 A1
(43) Pub. Date: Sep. 18, 2008

(54) MATERIAL AND A METHOD FOR REMOVING OXO-ANIONS AND METAL CATIONS FROM A LIQUID

(75) Inventors: Muhammad Nahidul Hasan, Delft (NL); Marinus Cornelis Maria Van Loosdrecht, Den Haag (NL); Wilfred Raymond Hagen, Oosterbeek (NL)

Correspondence Address: PEACOCK MYERS, P.C., 201 THIRD STREET, N.W., SUITE 1340, ALBUQUERQUE, NM, 87102, US

(73) Assignee: Technische Universiteit Delft, Delft (NL)

(21) Appl. No.: 12/046,667

(22) Filed: Mar. 12, 2008

Related U.S. Application Data
(63) Continuation-in-part of application No. PCT/EP2006/066277, filed on Sep. 12, 2006.

(30) Foreign Application Priority Data
Sep. 16, 2005 (EP) 050771047

(51) Int. Cl. C02F 1/62; C07K 14/79

(52) U.S. Cl. 210/684; 210/500.27; 210/685; 435/69.6

(57) ABSTRACT

A material for removing oxo-anions and metal cations from a liquid. This material is characterized in that it comprises hyperthermophilic ferritin. The present invention also relates to a method for removing at least one of oxo-anions and metal cations from a liquid. This method is characterized in that a material comprising hyperthermophilic ferritin is used as filter material. It is especially preferred that said liquid is comprised of water. The invention also relates to the use of hyperthermophilic ferritin for removing metal cations and oxo-anions from a liquid.

### CROSS-REFERENCE TO RELATED APPLICATIONS

This application is a continuation-in-part application of Patent Cooperation Treaty International Application Ser. No. PCT/EP2006/066277, entitled “A MATERIAL AND A METHOD FOR REMOVING OXO-ANIONS AND METAL CATIONS FROM A LIQUID”, to Technische Universiteit Delft, filed on Sep. 12, 2006, and the specification and claims thereof are incorporated herein by reference.

This application claims priority to and the benefit of the filing of European Patent Office Application Serial No. 05077104.7, entitled “A MATERIAL AND A METHOD FOR REMOVING OXO-ANIONS AND METAL CATIONS FROM A LIQUID”, filed on Sep. 16, 2005, and the specification and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

1. Field of the Invention (Technical Field)

The present invention relates to a material for removing oxo-anions and metal cations from a liquid. The invention also relates to a method for removing at least one of oxo-anions and metal cations from a liquid.

2. Description of Related Art

It is in the art known to remove oxo-anions and metal cations from a liquid. For example, several filtration systems are available, that make use of, among others, activated alumina, a granular ferric hydroxide, manganese dioxide coated sand or iron coated sand, polyethylene enhanced ultrafiltration and nano-particulate transition metal oxide. These systems are either inefficient at low contaminant concentration or they add new contaminants, microbes, etc. As a matter of fact, these known systems require many hours for removing the stated impurities from the liquid. Also, these known systems are capable of removing either metal cations or oxo-anions from the liquid.

Therefore, there is a need in the art for a material with which oxo-anions and metal cations can be removed from a liquid.

### BRIEF SUMMARY OF THE INVENTION

Therefore, the present invention aims at providing a material for removing oxo-anions and metal cations from a liquid that comprises a material that is capable of removing at least one of these contaminants.

Furthermore, the invention aims at providing a material that is capable of performing the filtration of a liquid in a much shorter time than the known materials.

Also, the invention aims at providing a material that is capable of removing contaminants at very low concentrations, preferably at concentrations much lower than is possible with the known materials.

### BRIEF DESCRIPTION OF THE SEVERAL VIEWS OF THE DRAWINGS

Not Applicable.

### DETAILED DESCRIPTION OF THE INVENTION

So as to obtain at least one of the above aims, the invention provides a material as mentioned in the preamble and that is characterized in that it comprises hyperthermophilic ferritin.

The material according to the invention is capable of filtering contaminants at very low substrate concentrations. Furthermore, it is much quicker than the available materials, seconds in stead of minutes to hours. Also, it is capable of removing both metal cations and oxo-anions in a single step.

As a matter of fact, the material according to the present invention is regenerable and bio-degradable.

According to another aspect of the present invention, a method is provided for removing at least one of oxo-anions and metal cations from a liquid, which method is characterized in that the material comprising hyperthermophilic ferritin is used as filter material. This method in accordance with the present invention provides the advantages as indicated above with respect to the material.

It is especially preferred that a method is performed for filtering water. According to this method, contaminant concentrations of metal cations and oxo-anions can be obtained that correspond or are lower than the current maximum contaminant level (MCL) of solutes in drinking water, suggested by the World Health Organization, and others.

According to a further preferred embodiment, a method is characterized in that said oxo-anions and metal cations are chosen from any of iron ions, phosphate, arsenate, vanadate, tungstate and molybdate ions. These ions can be removed efficiently with the present method.

It is further preferred that the method according to the invention comprises the step of performing a conversion of ferrous iron to ferric iron with the hyperthermophilic ferritin. Then, the iron ion is rendered insoluble, and as a matter of fact it will be precipitated in the protein shell of the ferritin protein.

Furthermore, it is preferred that said method comprises the step of performing an absorption of an oxo-anion in the protein shell of the ferritin material. It is especially preferred if a co-precipitation of the ferric iron and the oxo-anions in the protein shell is performed.

According to a further aspect of the present, invention, it. relates to the use of a hyperthermophilic ferritin for removing metal cations and oxo-anions from a liquid. It is especially preferred if this use is related to the removal of metal cations and oxo-anions from water, so as to obtain drinking water that fulfils the requirements of the World Health Organization, the United Nations and others.

Finally, the present invention relates to a method for producing a hyperthermophilic ferritin, comprising the steps of:
(a) Construction of a recombinant E. Coli strain containing structural gene of ferritin,
(b) over-production of the recombinant protein, and
(c) single step purification by heat treatment.

A specific advantage of the material according to the present invention is the ability to remove oxo-anions at very low concentrations, of below 10 ppb. The concentration obtainable with the present material is much beyond the capacity of the currently available technics.

Also, the ability to be sterilized repeatedly is an important advantage of the present invention. For, if the material according to the present invention would be contaminated with microbes, etc., it can be sterilized, so as to yield a clean and safe filtering material.

Sterilization may be performed in an autoclave at 120° C. for 20 minutes. No loss of activity is obtained by such method.

A material according to the present invention is bio-degradable. Bio-degration can be obtained by means of peptidase enzymes.

The hyperthermophilic ferritin that, is used in accordance with the present invention, is stable over a wide temperature range. It is especially capable to be used at a temperature of from 0° C. to 100° C.

In contrast to the materials known in the art, and that are used for removing metal cations or oxo-anions from a liquid, the present material is capable of removing both of these ions. Such is completely novel in the art.

The hyperthermophilic ferritin can be produced as follows:

### Constructing Recombinant E. Coli Strain:

The putative ferritin gene from the Pyrococcus furiosus cells was amplified by the PCR method using oligonucleotides 5′-CCATATGTTGAGCGAAAGAATGC-S′ as the forward primer and 5′-GTCGACT-TACTCCTCCCTG-3′ as the reverse primer. Then the gene was cloned into pCR 2.1-TOPO shuttle vector (Invitrogen) and transformed into competent E. coli DH5α Cells for further amplification. The chimeric vector was then isolated from the cells and sequenced to check the fidelity of the clone. The ferritin gene was then separated by Ndel and Sa1l (Roche) restriction digestion and recloned into expression vector pET24a (+) (Novagen). The resulting clone was then transformed into the competent BL21-CodonPlus (DE3)-RIL cells (Stratagene).

### Overexpression of the Recombinant Protein:

A single colony of the recombinant E. coli strain was inoculated in LB (Sambrook, et al ., 1989) media containing 50 mg ml⁻¹ of chloramphenicol and 20 mg ml⁻¹ of kanamycin and cultivated overnight aerobically at 310 K and 200 rev. min⁻¹. This pre-culture was transformed in TB medium (Sambrook, et al., 1989) in a 1:20 ratio and grown for 2 h (until it reaches the O.D₆₀₀ value of 0.5 or higher) and induced with 1 mM IPTG. Cells were then harvested by centrifugation after an additional 5 h of growth.

### Purification of Ferritin:

The cell pellet was washed and re-dissolved in 20 mM Tris-HCl buffer, pH 8. DNase and RNase were added to the solution in order to degrade the genetic elements and 0.5 mM PMSF was added to protect any protein degradation by internal protease activity. The cell suspension was then applied to a Cell Disruptor (Constant Systems) to break the cells. The cell-free extract was subjected to heat treatment (373 K, 30 min) and clarified by centrifligation. The resultant supernatant was concentrated over Amicon, YM-100, to give the final preparation of the protein.

The present invention is not limited to the description as given herein before. It is only limited by the appended claims.

What is claimed is:

1. A material for removing oxo-anions and metal cations from a liquid, the material comprising hyperthermophilic ferritin.

2. A method for removing at least one of oxo-anions and metal cations from a liquid, the method comprising the steps of providing such a liquid and filtering the liquid with a material comprising hyperthermophilic ferritin.

3. A method according to claim 2, wherein said liquid is water.

4. A method according to claim 2, wherein said oxo-anions and metal cations are selected from the group consisting of iron ions, phosphate, arsenate, vanadate, tungstate and molybdate.

5. A method according to claim 2, wherein the method additionally comprises the step of performing a conversion of ferrous iron to ferric iron with the hyperthermophilic ferritin.

6. A method according to claim 2, wherein the method additionally comprises the step of performing an adsorption of an oxo-anion in the protein shell of the ferritin material.

7. The use of a hyperthermophilic ferritin for removing metal cations and oxo-anions from a liquid.

8. Use according to claim 7, for removing metal cations and oxo-anions from water.

9. A method for producing a hyperthermophilic ferritin comprising the steps of:
(a) Construction of a recombinant E. Coli strain containing structural element of the ferritin gene,
(b) over-production of the recombinant protein, and
c) single step purification by heat treatment.
